(19) United States
(12) Patent Application Publication
(10) Pub. No.: US 2013/0289501 A1
(43) Pub. Date: Oct. 31, 2013

(54) SKIN TREATMENT PROCESS AND DEVICE

(71) Applicants: Arpita Bhattacharya, Bangalore (IN); Sudipta Ghosh Dastidar, Bangalore (IN); Alagirisamy Nethaji, Bangalore (IN); Rudra Saurabh Shresth, Bangalore (IN); Narayanan Subrahmaniam, Bangalore (IN)

(72) Inventors: Arpita Bhattacharya, Bangalore (IN); Sudipta Ghosh Dastidar, Bangalore (IN); Alagirisamy Nethaji, Bangalore (IN); Rudra Saurabh Shresth, Bangalore (IN); Narayanan Subrahmaniam, Bangalore (IN)

(21) Appl. No.: 13/995,217

(22) PCT Filed: Dec. 14, 2011
(86) PCT No.: PCT/EP2011/072675

§ 371 Date: Jul. 8, 2013

(30) Foreign Application Priority Data
Dec. 24, 2010 (IN) 3519/MUM/2010
Feb. 22, 2011 (EP) 11155302.0

(51) Int. Cl. A61H 33/02; A61M 35/00

(52) U.S. Cl. CPC A61H 33/027; A61M 35/00
USPC 604/290; 4/596; 4/616; 132/212

(57) ABSTRACT

The present invention is in the field skin treatment, in particular and deep pore cleansing and delivery of benefit agents deep inside the pores. The invention further relates to air-water jet devices for providing said skin treatment. Accordingly it is an object of the invention to provide for a device and process for deep pore skin cleansing, especially, a process for skin cleansing using reduced amounts of water. We have found that a skin cleaning device comprising an air-water jet, wherein the air and water are mixed outside the nozzle(s), provides improved cleansing of skin, including deep poor cleansing, with low usage of water.

### FIELD OF THE INVENTION

The present invention is in the field skin treatment, in particular and deep pore cleansing and delivery of benefit agents deep inside the pores. The invention further relates to air-water jet devices for providing said skin treatment.

### BACKGROUND OF THE INVENTION

Deep pore skin cleansing has been considered in the art and devices are available in the market.

Skin creams containing abrasive particles (also referred to as scrub creams) are widely available in the market mostly for the purpose of skin exfoliation. However, such creams are mostly effective for the removal of dead skin cells, rather than deep pore skin cleansing.

Ultrasonic skin cleaning devices, such as the Mia™ Sonic skin cleansing system by ClariSonic are also commonly available. Ultrasonic devices are said to release dirt by Ultra sound waves. Ultrasound wave based devices primarily work by generating local heating into the deeper tissues and especially the collagen. It also produces high speed mechanical vibrations which act on the tissues like micro massage facilitating blood flow and improved circulation. Cavitation is another benefit which countless microscopic oxygen droplets of oxygen due to the vibration process. However, ultrasonic waves dampen significantly in contact with skin and the cleaning efficiency is not sufficient.

Similarly devices utilising vacuum for cleaning pores are available, such as the GEZATONE® Vacuum Skin Cleansing Device, by Gézanne. Vacuum devices are intended to physically suck the dirt from the pores. However, simple suction based devices are not efficient in removing dirt from deep pores. Moreover the negative pressures one would need to remove entrapped dirt particles are usually large.

Other water jet devices and air-water jet devices have been disclosed in the art, but they are found to either use too much liquid to be useful in solving for the present problem such as JP 10 305078 A, JP 2004 275701 A, or uses and atomistation system that does not provide adequate impact to be suitable for deep pore cleansing and treatment, such as EP 1 116 521 A2.

An easy-to-operate device for deep pore cleansing remains to be desired.

Accordingly it is an object of the invention to provide for a device and process for deep pore skin cleansing.

It is a further object to provide a device and process for the delivery of benefit agents to the skin.

It is yet a further object to provide a process for skin cleansing using reduced amounts of water.

In our co-pending application WO2009/103595, a cleaning device is disclosed that comprises a novel kind of air-water jet and methods for using the same for cleaning substrates, such as fabric articles.

Surprisingly it has been found that a skin cleaning device comprising an air-water jet, wherein the air and water are mixed outside the nozzle(s), provides improved cleansing of skin, including deep poor cleansing, with low usage of water.

### SUMMARY OF THE INVENTION

Accordingly, the present invention provides a process for treating a keratinous substrate with a cleaning device comprising an air-water jet device comprising two nozzles wherein a first nozzle is in fluid communication with a feed liquid source; and a second nozzle connected to a source of compressed air.

In the context of the present invention by skin is meant any keratinous substrate (also referred to as surface), including but not limited to skin, hair and nails. These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

### DETAILED DESCRIPTION OF THE INVENTION

The present invention provides deep pore skin cleansing and delivery of benefit agents to skin pores by means of an air-water jet stream as generated by and air-water jet device.

### Air-Water Jet Device

The air-water jet device comprises two nozzles wherein a first nozzle is in fluid communication with a feed liquid source; and a second nozzle connected to a source of compressed air. The air-water jet device is incorporated into a skin applicator.

The air-water jet according to the invention is further disclosed in WO02009/103595 (Unilever), incorporated herein by reference.

The liquid source may be any water source, either provided to the air-water jet device straight from the water mains, through a pump, through a pressured container holding the water or by any other means, or even by gravity (i.e. by placing the water reservoir above the height of use of the air-water jet.

The feed liquid may be any liquid. For regular skin cleansing an aqueous composition is typically preferred. The nozzle for the liquid is called water nozzle herein below, but it is understood that the water nozzle may pass water or any other liquid, including aqueous liquids and other skin treatment composition optionally comprising benefit agents.

Similarly, the air source may be any air source, either provided through a compressor, separate from, or built into the skin applicator cleaning device, or through a compressed air line, such as often available in hospitals and in dental clinics.

Both, the first nozzle, (water nozzle) and the second nozzle (air nozzle) are positioned relative to an imaginary central axis (NOR). The first nozzle is positioned at an angle (α) of between 1 and 60°, preferably between 10° and 30° relative to the central axis; and the second nozzle is at an angle (φ) of between 1 and 45°, preferably between 15° and 30° relative to the central axis.

The mouth of the second nozzle is positioned more forward in the direction of the flow along the direction of the central axis than the mouth of the first nozzle, wherein the offset (OS) distance between the mouth of the first nozzle and the second nozzle is between 0.5 and 5 mm in said direction, preferably 1-3 mm.

The best results are obtained when the first nozzle has an opening of between 0.05 and 10 mm², preferably even at least 0.2 mm², and not more than 7 mm², more preferably not more than 5 mm² or even less than and 3 mm². Similarly, the opening of the second nozzle is preferably between 0.2 and 3 mm².

For nozzles with a circular opening, the diameter of the first nozzle is preferably between 0.25 and 3.5 mm, preferably at least 0.5 mm, but preferably not more than 3 mm, more preferably not more than 2.5 mm, or even less than 3 mm; while the diameter of the second nozzle is preferably between 0.5 and 2 mm. The scope of the present invention further includes configurations comprising two or more water nozzles directed at a single air nozzle. Although this adds to the complexity of the device, which is generally not preferred, it provides the additional benefit of point of action mixing or reacting different or incompatible ingredients.

Additionally different nozzle geometries are contemplated, including but not limited to circular, square, rectangular and oval openings, for either one or both nozzles. For larger surfaces, a slit type (oval or rectangular) air nozzle and a similarly dimensioned water nozzle, or multiple water nozzles are preferred, more preferably the ratio between the longest side-to-side distance and the shortest side-to-side distance of the oval or rectangular slit, is between 2:1 and 20:1, still more preferably between 2:1 and 10:1. Cross or star shaped air nozzles with one or more water nozzles being positioned between the extending parts (i.e. in the indentations) are also contemplated in this context.

Without wishing to be bound by a theory, it is thought that the present invention derives its performance from the positioning of the nozzles relative to the imaginary axis and the offset of the water nozzle (first nozzle) relative to the air nozzle (second nozzle). Because of this positioning, the feed liquid coming from the water nozzle forms a film around the air nozzle, and because of this, it gives a finer spray at a lower liquid-to-air ratio (i.e. using less liquid). The air flow from the air nozzle is thought to create a local under-pressure that ensures that the liquid is driven in the direction of the air nozzle along the air nozzle tip, regardless of in which direction the nozzle is pointed. Furthermore, the liquid flow is not affected by the air pressure due to the separation of the air and water nozzle openings, which is a common problem with internal mix nozzle designs.

It is further preferred that the nozzles are close to the surface of the substrate when the device is in operation, preferably the distance of the air nozzle is less than 1 cm away from the surface, more preferably, less than 5 mm, still more preferably less than 3 mm, even more preferably less than 2 mm, yet more preferably less than 1 mm, or even less than 0.5 mm away from the surface.

It is therefore preferred that the liquid:air ratio is between 10:90 and 1:9999, more preferably less than 5:95, still more preferably less than 4:96, even more preferably less than 3:97, less than 2:98 or even less than 1:99, while the ratio is preferably higher than 3:9997, more preferably higher than 5:9995.

It is further preferred that there is only a short distance between the opening of the water nozzle and the side of the air nozzle, this distance is preferably less than 2 mm, more preferably less than 1 mm, or even less than 0.5 mm. It is most preferred that the opening of the water nozzle is touching the air nozzle.

It is preferred that the air nozzle does not co-axially surround the water passage. It is also preferred that the water nozzle does not co-axially surround the air nozzle.

The air pressure of the air source is preferably in the range of 1 to 5 bar. The air preferably has a velocity of greater than 80 m/s at the exit of the nozzle (the nozzle opening), preferably greater than 120 m/s, more preferably greater than 180 m/s, and most preferably greater than 250 m/s. Although the invention would work up to very high air velocities, it is preferred for constructional reasons and convenience for the user, that the air velocity is less than the speed of sound (i.e. less than 334 m/s). Depending on the nozzle diameter, the airflow rate is preferably between 3 and 50 l/min, preferably more than 5 l/min or even more than 10 l/min. The air flow rate is preferably less than 40 l/min, more preferably less than 30 l/min or even less than 25 l/min.

The liquid flow rate is typically between 2 and 100 ml/min, preferably more than 5 ml/min or even more than 10 ml/min, while the liquid flow rate is preferably less than 80 ml/min, more preferably less than 50 ml/min, or even less than 40 ml/min.

### Configuration

The air and/or liquid sources may be incorporated into the device, or be fitted in a separate unit. In the latter case, a separate unit comprising a compressor, a compressed air cartridge or cylinder, or another source of air and/or a liquid reservoir, optionally connected to the water mains, is provided. The unit is connected to a hand held device by means of a tubing as air line and/or water line.

### Applicator Head

The device preferably comprises an applicator head and a handle. The applicator head typically comprises the air-water jet device. The use of more than one air-water jet devices is also contemplated.

The device of the present invention may further incorporate other cleaning features such as bristles, scrubbers and/or massaging elements. These elements are preferably positioned in the brush head.

For hair application, including scalp pore cleansing and deposition of benefit agents to the scalp, as well as low-water hair washing, a configuration of the device in the form of a comb comprising multiple nozzles is also contemplated in the context of the present invention. Alternatively, an applicator head comprising the air-water jet and comb elements is also considered.

The applicator head may further be electrically operated. In this respect, the applicator head may be driven by an electric motor incorporated into the handle of the device. The motor may move the head back and forth linearly in the direction of the handle, back and forth transverse at a 90° angle with said direction, back and forth over an angle of 1-180°, preferably 1-90° or even 1-45° around an axis in the direction of the handle, in a circular motion around an axis transverse to the direction of the handle, or back and forth over an angle of 1-180°, preferably 1-90° or even 1-45° around an axis transverse to the direction of the handle; or a combination thereof. In all the above configurations, the air-water jet and the optional bristles and or massaging elements are preferably pointing in a direction that is transverse to the handle of the device.

The device may further comprise an air compressor as air source. The compressor may be built into the handle of the device, or provided as a separate device that is connected to the air-water jet by means of a tube. The compressor preferably provides at least 1 bar pressure and not more than 5 bar, preferably less than 4 bar. Thus, very low power compressors, typically in the range of 0.05 to 1 HP, can be used to achieve the above specifications. Due to a pressure drop in the tubing and the device, the pressure at the air nozzle will preferably be in the range of 1 to 4 bar, more preferably 2 to 3 bar. A device with a means to set the pressure is also contemplated; in this case the user is, for instance, able to choose between skin surface cleansing, or deep pore cleansing.

The liquid source may be the water mains, i.e. directly connected to the faucet, or be in the form of a separate reservoir. The pressure on the liquid source for use with the cleaning device may be relatively low, preferably at least 0.05 bar, more preferably at least 0.1 bar, but preferably not more than 3 bar, more preferably less than 2.5 bar, still more preferably less than 2 bar.

When a separate reservoir is used as liquid source, said reservoir may be filled with water only, a cleaning composition, a composition comprising benefit agents.

The liquid reservoir may be placed above the level of use of the cleaning device, such as to provide pressure, or may be pressured separately. When pressured separately, it is especially preferred that the reservoir is pressurised with compressed air from the compressed air source.

### Treatment Compositions

Different kinds of treatment compositions are envisaged within the scope of this invention. Although regulatory requirements may prescribe that the concentrations of the compositions applied through the air-water jet are limited, the concentrations may exceed such levels for the effect of operation.

### Deep Pore Cleansing

Deep pore cleansing compositions are typically used for cleaning skin and/or scalp pore cleansing.

Deep pore cleansing compositions may comprise of surfactants, soaps, solvents, absorbing particles like clays, and polymers.

### Skin Surface Cleansing

The device and process of the invention enable the user to wash their skin with low amounts of water.

Skin cleaning compositions typically comprise a liquid continuous phase and one or more benefit agents. Most typically, the liquid continuous phase comprises water, usually as the major component, making up greater than 50%, or even greater than 90%, by weight of the feed liquid.

Skin cleaning compositions to be used in the present invention, are typically aqueous, but may be solvent based.

The compositions may further comprise a surfactant. Surfactants typically used in a face wash composition are Sodium Lauryl Sulfate, Cetyl betaine, Alkali metal (such as potassium) soaps of fatty acids like Myristic acid, Lauric acid, Palmitic acid, steatic acid etc, Sodium cocoamphoacetate, Disodium, Laureth sulfosuccinate, ethylhexyl stearate, Glyceryl stearate, Cetyl palmitate, Decyl Oleate, SLES (e.g. SLES-3EO).

The composition may further comprise natural extracts, solvents, humectants, cellulose derivates, structuring polymers, preservatives etc.

Preferred natural extracts appreciated by the consumers include Azadirachta indica, Acorus calamus, Glycyrrhiza glabra, Emblica officinalis, and green tea extracts.

Preferred skin benefit agents suitable for use in the present invention include anti acne actives (e.g. Salicylic acid) and anti ageing actives (e.g. Vitamins C, Vitamin E, Vitamin A, Alpha Hydroxy Acids, Glycolic acids, N-6 furfuryladenine) and skin lightening agents (eg. Ethyl Resorcinol, Niacinamide)

Preferred skin cleansing compositions are fully or partially solvent based. Typical solvents used in such compositions include, alcohols, silicone oils, hydrocarbon oils, and oils comprising of fatty acids and/or triglycerides of fatty acids, including oils of natural origins (e.g. olive oil)

### Hair Wash (Shampoo) and Treatment

By hair wash and treatment is meant both the cleansing of hair, especially low water washing of hair, and deposition of benefit agents to the scalp, such as anti-dandruff agents.

Hair washing compositions may comprise soaps, surfactants, polymers and actives like ZPT (zinc pyrithione), selenium sulphide, octopirox, ketoconazole, climbazole and salicylic acid.

Other treatment agents which are considered for scalp treatment in the context of the present invention are Minoxidil for topical administration, Finasteride, protease inhibitors connected with hair loss, such as Trichogen, Climbazole and Zinc gluconate.

In one embodiment the hair and scalp treatment compositions may comprise anti-dandruff agents. Dandruff (Pityriasis simplex capillitii) is generally caused by skin micro-organisms (specifically Malassezia yeasts). Typical anti-dandruff agents are salicylic acid, sulfur based compositions, keratinization regulators, e.g. zinc pyrithione (ZPT), tar based compositions, steroids, e.g. corticosteroids, selenium sulfide imidazole antifungal agents, e.g. ketoconazole, hydroxypyridones and naturopathic agents

### Operation

While using the device, the air-water jet may be used continuously, or discontinuously. One way of operation that is considered is to use the air-water jet during part of the operation. In another embodiment, the air-water jet is used in the first part of the cleaning process for cleaning and run with only the liquid flow or the liquid flow and low air flow to deposit a benefit agent to the skin or scalp.

Preferred benefit agents are ZPT, selenium sulphide, octopirox, ketoconazole, climbazole and salicylic acid, Minoxidil for topical administration; Finasteride and proteases inhibitors connected with hair loss, e.g. Trichogen; Climbazole and Zinc gluconate.

In another embodiment the air-water jet is operated in a pulsed mode i.e. the air flow is controlled in an on-off fashion over time. In yet another embodiment the handheld device is fitted with a push button to switch the air-water jet on or off while cleansing and treatment skin (also including hair, scalp and other keratinous surfaces are defined herein above).

In any of the discontinuous operations, it is preferred to open and shut the air and/or liquid lines with a suitable solenoid valve.

A valve system may also be used to open the liquid and/or air lines when the device is in operation, while shutting the liquid and/or air lines when the device is not in use.

The air-water jet device may be used to clean the skin operating with just water, or with a commercially available skin cleansing composition.

Even better results are obtained when applying a conventional skin cleansing composition to the skin prior to cleansing with the air-water jet device of the present invention.

Accordingly, the invention provides process for treating a keratinous substrate with a cleaning device comprising a an air-water jet device comprising two nozzles wherein a first nozzle is in fluid communication with a feed liquid source; and a second nozzle connected to a source of compressed air.

Ideally, but not necessarily both nozzles are positioned relative to a central axis, wherein the first nozzle is at an angle of between 1 and 60° relative to the central axis; and the second nozzle is at an angle of between 1 and 45° relative to the central axis. Preferably, the air nozzle does not co-axially surround the water passage and wherein the mouth of the second nozzle is positioned more forward in the direction of the flow along the direction of the central axis than the mouth of the first nozzle, wherein the offset distance between the mouth of the first nozzle and the second nozzle is between 0.5 and 5 mm in said direction.

The liquid passage may be filled with a cleansing composition or a benefit agent. The process of the invention enables deposition of the cleaning composition or benefit agent deep into the pores of the skin, and onto hair or scalp through the first nozzle (connected to the liquid source). Without wishing to be bound by a theory, it is thought that deep pore cleaning, delays the time for skin to become and feel greasy again. Surface cleansing typically only removes the fatty and greasy material on the outside of the skin. After washing, the skin then feels clean until fatty material from the skin pores have made the surface feel greasy again. If the fatty material is removed also from deep inside the pores, it is thought that this delays the time for the skin to feel greasy again.

Similarly, the process may optionally be used to deposit a benefit agent onto the skin selected from anti acne actives, anti ageing actives, skin lightening agents or combinations thereof.

Similarly, the process may optionally be used to deposit a benefit agent onto the hair or scalp, selected from Minoxidil, Finasteride and protease inhibitors, or combinations thereof.

Similarly, the process may optionally be used to deposit an anti dandruff agent onto the hair or scalp, selected from salicylic acid, sulfur based compositions, keratinization regulators, tar based compositions, steroids, selenium sulfide imidazole antifungal agents, hydroxypyridones and naturopathic agents, or combinations thereof.

### EXAMPLES

The invention will now be demonstrated by means of the following non-limiting examples.

### Example 1

### In-Vitro Test for Deep Pore Cleansing

### 1. Preparation of Model Skin Capillaries (Tuneable Capillaries)

Copper wires of 100 μm diameter were hung vertically with the help of a fixture. The loose ends of the wires were positioned inside a trough. The other end of the trough was fitted with a movable piston. Molten paraffin wax was poured inside the trough and was subsequently allowed to solidify. The copper wires were pulled out from the wax slab, thereby resulting in the formation of capillaries inside the wax slab. The trough piston was pushed out to remove the wax slab. The slab was further trimmed using a Leica Microtome to a height of 3 mm.

The slab was placed in a pool of liquid model sebum so that the liquid rises inside the capillary. The model sebum used was olive oil. The model sebum was tagged with 16 μm Nile red as colour indicator. The slab was removed from the pool and dipped inside a thin film of molten wax to seal one end of slab. Thus microwells filled up with liquid sebum were prepared and used as model skin capillaries.

2 Cleaning with the Air-Water Jet Device (According to the Invention)

1% SLES-3EO (sodium lauryl ether sulphate ethoxylated with 3 moles of ethylene oxide per mole) in water was sprayed through the air-jet device at 3 bar gauge air-pressure and 25 mL/min liquid flow rate for 10 sec. The nozzle diameter is 0.5 mm.

### 3 Manual Cleaning (Comparative Example)

1% of SLES 3EO was applied without the compressor for 10 secs with a flow rate of 25 ml/min. The cleaning was performed by rubbing five times clock wise and five times anti clock wise.

### 4 Evaluation of Cleansing

The model sebum removal was evaluated using a LASER Scanning Confocal Microscope from Leica (Model DMR), fitted with a TCS SP2 scanner head. The excitation wavelength was 514 nm generated using an Argon ion LASER. The emission wavelength range was set to be between 530 nm to 625 nm. The Airy disc was set to be 180 μm. The imaging was done through optical slicing for every 10 microns.

Table 1 below demonstrates the intensity vs dept profile of manual treatment (comparative) and air-water jet application (example according to the invention). A greater intensity means less clean. For a completely cleaned surface the intensity reading is zero.

TABLE 1
Results

| | |
|---|---|
| Comparative example | Example |

| Depth | Before | After | Before | After |
|---|---|---|---|---|
| (micron) | treatment | Treatment | Air-Jet | Air-Jet |
| 0 | 255 | 255 | 255 | 0 |
| 9 | 255 | 255 | 255 | 0 |
| 18 | 255 | 255 | 255 | 0 |
| 26 | 253.6 | 255 | 255 | 0 |
| 35 | 253.4 | 255 | 255 | 0 |
| 44 | 253.3 | 255 | 255 | 0 |
| 53 | 252.2 | 255 | 255 | 0 |
| 61 | 251.1 | 255 | 255 | 0 |
| 70 | 249.0 | 255 | 255 | 0 |
| 79 | 231.4 | 255 | 255 | 0 |
| 88 | 220.9 | 255 | 242 | 0 |
| 96 | 199.8 | 255 | 234 | 0 |
| 105 | 161.9 | 248.6 | 185 | 0 |
| 114 | 137.1 | 246.8 | 165 | 0 |
| 123 | 135.1 | 244.2 | 160 | 0 |
| 131 | 127.6 | 202.4 | 156 | 0 |
| 140 | 113.5 | 199.2 | 145 | 0 |
| 149 | 91.9 | 198.6 | 123 | 0 |
| 158 | 54.6 | 142.6 | 119 | 0 |
| 166 | 42.2 | 135.2 | 76 | 0 |

### Example 2

### Air-Jet on Transpore Tape to Evaluate Foundation Removal

Glass slides (Blue Star micro slides, Polar Industrial Corporation) were taken, rinsed with DI water and wiped dry using tissue paper. Transpore (ex 3M) tape having a width of 1 inch (=2.54 cm), was stuck on the glass slide and a 3 cm 2 area was marked on it for soiling. The soiling was done using cosmetic foundation (Revlon New Complexion—02 Creamy Peach Beige). 5 μL of it was applied horizontally and spread using parafilm followed by 5 μL vertically to achieve uniform coverage. The slides were kept for drying for 1 hour.

Cleaning with the air-water jet was done at an air pressure of 3 bars and liquid flow rate of 25 mL/min. The nozzle diameter is 0.5 mm

The cleaning liquid used was 1 g/L SLES-3EO solution in DI water. The cleaning was done for different times (15 s, 30 s, 1 min, 2 min, 5 min, 10 min). Cleaning manually was done by rubbing with finger for the above given times. The solution used was an 11.4 g/L solution of SLES-3EO in DI water and the surfactant concentration was matched with the respective air-jet experiments as given in Table 2. The experiment was repeated twice more to get three data points for each set.

The slides were rinsed by pouring 50 mL DI water on each and then kept in a hot air oven (PSM/HAO/06, P.S.M. Scientific Instruments (P) Ltd.) at 40° C. for 3 hours to dry them. Finally reflectance data (R460*) was collected using a reflectometer (Gretag Macbeth™ Color-Eye 7000 A) as given in Table 2. A white paper was kept at the back of the slides to minimise error due to the transparency of the slides.

TABLE 2
Results

| Air-jet | | | | Manual | | | |
|---|---|---|---|---|---|---|---|
| Time | | | | Time | | | |
| (sec) | R460* | Average | Std Dev | (sec) | R460* | Average | Std Dev |
| 15 | 29.65 | 34.16 | 4.08 | 15 | 31.74 | 30.864 | 1.77 |
| 15 | 37.58 | | | 15 | 28.82 | | |
| 15 | 35.24 | | | 15 | 32.03 | | |
| 30 | 41.89 | 42.01 | 1.83 | 30 | 35.79 | 31.996 | 3.29 |
| 30 | 40.24 | | | 30 | 30.13 | | |
| 30 | 43.89 | | | 30 | 30.07 | | |
| 60 | 46.49 | 42.20 | 4.97 | 60 | 34.48 | 33.013 | 1.65 |
| 60 | 43.36 | | | 60 | 33.33 | | |
| 60 | 36.76 | | | 60 | 31.23 | | |
| 120 | 63.01 | 60.02 | 2.77 | 120 | 40.82 | 35.880 | 4.33 |
| 120 | 59.50 | | | 120 | 34.10 | | |
| 120 | 57.55 | | | 120 | 32.72 | | |
| 300 | 60.10 | 56.79 | 3.48 | 300 | 38.36 | 35.686 | 2.62 |
| 300 | 57.10 | | | 300 | 33.13 | | |
| 300 | 53.16 | | | 300 | 35.56 | | |
| 600 | 60.05 | 60.30 | 2.44 | 600 | 31.61 | 40.592 | 7.78 |
| 600 | 57.99 | | | 600 | 45.16 | | |
| 600 | 62.85 | | | 600 | 45.01 | | |

The table above shows that the cleaning performance with the air-water jet device according to the invention is superior to the manual washing, even though the manual washing was done with a higher surfactant concentration.

### Example 3

### Air-Jet on Pig Skin to Evaluate Foundation Removal

Pig's skin is cut into 5 cm×5 cm area and the hairs on the skin are cut short. An area of 3.5 cm×2.5 cm is marked on it. 0.02 mL of foundation (Revlon New Complexion—02 Creamy Peach Beige) is dispensed on this area and spread uniformly. This was then kept in refrigerator at −4° C. for an hour. Following this cleaning of the soiled area is done using air-jet and face wash (Ponds Perfect Matte). With air-jet cleaning was done for 15 seconds, pressure was 3 bar and the liquid flow rate was 25 mL/min. The cleaning solution used was 5 g/L SLES-3EO solution in DI water. Manual cleaning was done with finger by rubbing 5 times in clockwise and 5 times anti-clockwise. Cleaning was done with 0.15 g of face wash. The cleaned skin pieces were then rinsed by pouring 50 mL DI water. Reflectance measurements were done using a hand held reflectometer (Minolta 2600D). A black circular paper of width 0.6 cm and diameter 1 cm was kept in between the reflectometer (set to CIE-L*a*b* reading) and skin to avoid contact of the device with the pig's skin. The result is given in Table 3 below.

TABLE 3
Results

| Experi- | l* | | | a* | | | b* | | |
|---|---|---|---|---|---|---|---|---|---|
| ment | Initial | Final | Δl* | Initial | Final | Δa* | Initial | Final | Δb* |
| Foun- | 24.7 | 14.9 | −9.8 | 4.5 | 0.7 | −3.8 | 7.9 | 1.3 | −6.6 |
| dation | | | | | | | | | |
| (Face | | | | | | | | | |
| Wash) | | | | | | | | | |
| Foun- | 33.7 | 15.3 | −18.4 | 7.8 | 1.6 | −6.2 | 14.2 | 3.1 | −11.1 |
| dation | | | | | | | | | |
| (air-jet) | | | | | | | | | |

| | | | |
|---|---|---|---|
| Bare | 16.1 | 1.3 | 2.8 |
| Skin | | | |
| Area 1 | | | |
| Bare | 12.1 | 1.1 | 1.9 |
| Skin | | | |
| Area 2 | | | |

The table above shows that the cleaning performance with the air-water jet device according to the invention is superior to the manual washing.

### Example 4

### In-vivo Demonstration

The in-vivo of efficacy of the air-water jet device is demonstrated for deep pore cleansing as compared with direct application.

The air-water jet device is expected to be efficient in deep pore cleaning of keratinous substrates and may be useful to reduce the breakout of acne. To test the efficacy of the cleansing efficacy of this device, re-greasing measurement of sebum on forehead after cleansing would be an extremely useful determination in estimating the expectation of acne formation.

### Objective

The objective of the clinical study of this example was to determine if an air-water jet and cleansing composition has an added deep pore cleansing benefit as compared to the cleansing composition alone. The cleansing efficacy is assessed by measuring the re-greasing rate of sebum on forehead. The assumption is that if the air-water jet and the cleansing composition cleanses deeper in the pore, it will take a longer time for the sebum secreted from the sebaceous gland to reach the skin surface. By measuring skin surface sebum with a Sebumeter (ex Courage-Khazaka, China), the less sebum is expected on the site treated with air-water jet+cleansing composition when compared to the site treated with the cleansing composition only.

### Protocol

The study is performed as a double-blinded test, as a half forehead sebum re-greasing study with 20 male subjects. The measurements were repeated on the male subjects for two days. Hence the results obtained are an average of forty data points. The test subjects were asked to cleanse their forehead and face with a commercial soap bar three hours before coming to the clinical centre on each of the two visit days. They were not allowed to use any other product on their forehead during the duration of the study. The foreheads of the volunteers were marked at the two treatment areas separated by approximately 1 cm, one on each side of the forehead. Each square area is 3×3 cm 2 and further divided into four 1.5×1.5 cm 2 sub-sites. These sub-sites are used for taking sebum measurements at different time points (½, 1, 1½, and 2 hours) after cleansing. The time points to the locations are randomized within one cleansing area and symmetrically matched on the other cleansing area. After acclimation of the volunteers for twenty minutes, a Sebumeter (ex Courage-Khazaka) reading is taken on each of the 8 sub-sites and recorded as a reference sebum level. Then, one side of the subject's forehead was washed with a cleansing composition alone and the other side with cleansing composition+air-water jet according the procedures described below. Cleansing composition-only and cleansing composition+air-water jet treatments are evenly distributed between left and right sides of the forehead among test subjects. The entire procedure completed on day 1 is repeated on day 2 by switching the 2 treatments between the left and right sides of the forehead.

### Cleansing Procedures

During the study with the cleansing composition alone the study personnel dispensed approximately 0.2 ml of the test cleansing composition on a gloved hand washed the half head for 15 seconds, rinsed the side for 15 seconds and pat-dried with a paper towel. For the cleansing composition+air-water jet studies the study personnel operates the air-water jet device with the following parameters of pressure (3 bar gauge pressure), flow rate 25 ml/min and time of operation of 15 sec for the 3×3 cm 2 area. The number of sweeps of the air-water jet device was approximately ten times. After was the site was rinsed for 15 seconds and pat-dried with a paper towel. For both the studies the concentration of cleansing composition used was a 1% SLES-3EO solution in water.

### Results

The sebum measurements at various time points ate presented in the table below. The numerical indicate comparisons between treatments and are reported as change from baseline sebum measurements at each time point using paired t-test. The star indicates differences which are significant at 95% confidence limits. The results indicate that air-water jet+cleansing composition is significantly better in reducing sebum regeneration after 2 hours and a much efficacious cleansing composition as compared to a direct application.

### Results

| | N = 22 | 0.5 Hours | 1 Hours | 1.5 Hours | 2 Hours |
|---|---|---|---|---|---|
| Day 1 | SLES3EO | 32 | 58 | 83 | 106* |
| | Air-water jet + | 27 | 55 | 74 | 90* |
| | SLES3EO | | | | |
| Day 2 | SLES3EO | 25 | 53 | 79 | 118* |
| | Air-water jet + | 28 | 60 | 77 | 101* |
| | SLES3EO | | | | |

The results in the table above show that the air-water jet device has a higher efficacy, indicating better deep pore cleansing, than regular cleaning methods.

### Example 5

### Surface Cleansing Efficacy

This example was done in a randomized single-blind cleansing study with 20 subjects. Subjects have refrained from using any creams, lotions, moisturizers, sunscreens and washing appliances on the test sites up to 3 days prior to enrolment. This was a onetime wash study in which 8 test sites (4 per arm) were evaluated. Sites measuring 3.5 cm×2.5 cm (1.5in×1in) were demarcated on the arms using a skin safe marker. Following a pre-cleansing with an alcohol wipe and a 2 minute wait, study personnel have obtained baseline Chromameter CR-10 (trademark ex Konica-Minolta) measurements of subjects' test sites. This was followed by application of commercially available make-up product (brown foundation) to the test sites. The make-up tested was a marketed foundation. The tests were conducted in a completely randomized fashion. After make-up application and ten minutes of drying time, digital photographs were taken and Chromameter measurements of the test sites (with make-up on the skin) were made. The makeup was removed by application with the designated products. Following product application, the skin was dried for 10 minutes after which, digital photographs of the sites and final Chromameter measurements were taken.

Three tests were compared.
Example A: The Air-Water jet cleansing using 0.6% of SLES-3EO surfactant in water only.
Example B: Cleansing with a commercially available face wash composition (Ponds Perfect Matte Oil Control Cleansing Foam) only
Example C: First a pre treatment with the face wash composition, followed by treatment with the Air-Water jet cleansing using 0.6% of SLES-3EO surfactant in water.
The cleaning performance, measured as Delta-E was calculated by the conventionally known CIE-L*a*b* method.

For each of the two make-up products, the data was analyzed independently. A composite measure was compared to provide an indication of the average effect across a range of make up type. For each make up type and the composite measure, between treatment comparisons were conducted on the percent makeup removed using a 2-tailed paired t-test (at the 95% confidence level).

### Results:

| Test Formulations | Average Percentage cleaning (Delta-E) |
|---|---|
| Example A | 42 |
| Example B | 19 |
| Example C | 54 |

As demonstrated by the table above, just the face wash, or just the air water jet treatment do not give adequate cleaning, whereas the combined effect is superior to both.

### Example 6

### Evaluation of Exfoliation Efficacy

This example was done in a randomized single-blind cleansing study with 20 subjects. Subjects have refrained from using any creams, lotions, moisturizers, sunscreens and washing appliances on the test sites up to 3 days prior to enrolment. This was a onetime wash study in which 8 test sites (4 per arm) were evaluated. Sites measuring 3.5 cm×2.5 cm (1.5in×1in) were demarcated on the arms using a skin safe marker.

Two sets of controls were included: gelled-water and an untreated control. Eight nickel-sized circles (˜3.46 cm²) were drawn on the arms using a skin safe marker containing gentian violet dye. Following 10 minutes of drying, the sites were rinsed to remove any excess dye. The dye was removed by product application. Following 10 minutes of drying time, D-squame tapes will be applied to the sites, removed and placed on D-squame cards. Up to three Chromameter CR-10 measurements of the D-squames on the cards were taken for Control, Untreated and after treatment.

Three test setups were compared

Example D: The Air-Water jet cleansing using 0.6% of SLES-3EO surfactant in water only.
Example E: Cleansing with a commercially available face wash composition only
Example F: First a pre treatment with the face wash composition, followed by treatment with the Air-Water jet cleansing using 0.6% of SLES-3EO surfactant in water.

The cleaning performance, measured as Delta-E was calculated by the conventionally known CIE-L*a*b* method. The percentage removal/exfoliation is calculated as follows:

### Before=Untreated,

Control=washed with gelled water and dried,
After=after product application and dried.

$$\frac{\sqrt{{( {L}_{Control} - {L}_{After} )}^{2} + {( {a}_{Control} - {a}_{After} )}^{2} + {( {b}_{Control} - {b}_{After} )}^{2}}}{\sqrt{{( {L}_{Before} - {L}_{Control} )}^{2} + {( {a}_{Before} - {a}_{Control} )}^{2} + {( {b}_{Before} - {b}_{Control} )}^{2}}} * 100$$

Where,

L=Average L* reading
a=Average a* reading
b=Average b* reading

Product comparisons were made on the percent removed using paired t-tests and ANOVAs. Significance was generally determined with the p-value set at less than or equal to 0.05.

| Test Formulations | Average Percentage Removal |
|---|---|
| Example D | 65 |
| Example E | 40 |
| Example F | 80 |

As demonstrated by the table above, just the face wash, or just the air water jet treatment do not give adequate exfoliation, whereas the combined effect is superior to both.

### Example 7

### Deep Pore Cleansing

This example is done in the same way as Example 4.

Two test setups are compared

Example G: The Air-Water jet cleansing using 0.25% of SLES-3EO surfactant in water only.
Example H: First a pre treatment with the Ponds face wash composition, followed by treatment with the Air-Water jet cleansing using 0.25% of SLES-3EO surfactant in water.

The results are given below.

### Result

| | 5 min | 1.5 Hours | 2 Hours | 2.5 Hours |
|---|---|---|---|---|
| Example G | 4 | 56 | 90 | 120 |
| Example H | 3 | 52 | 77 | 109 |

The results in the table above show that the combined effort of face wash pre-treatment and air-water jet device has a higher efficacy, indicating better deep pore cleansing, than the air-water jet alone.

1. A process for treating a keratinous substrate with a cleaning device comprising
a) an air-water jet device comprising two nozzles wherein:
i) a first nozzle is in fluid communication with a feed liquid source; and
ii) a second nozzle connected to a source of compressed air characterised in that both nozzles are positioned relative to a central axis, and wherein
b) the first nozzle is at an angle of between 1 and 60° relative to the central axis; and
c) the second nozzle is at an angle of between 1 and 45° relative to the central axis; and
wherein the air nozzle does not co-axially surround the water passage and wherein the mouth of the second nozzle is positioned more forward in the direction of the flow along the direction of the central axis than the mouth of the first nozzle, wherein the offset distance between the mouth of the first nozzle and the second nozzle is between 0.5 and 5 mm in said direction.

2. A process according to claim 1, wherein a benefit agent is deposited to skin, hair or scalp through the first nozzle.

3. A process according to claim 1, wherein the benefit agent deposited onto the skin and is selected from anti acne actives, anti ageing actives, skin lightening agents or combinations thereof.

4. A process according to claim 1, wherein the benefit agent deposited onto the hair or scalp and is selected from Minoxidil, Finasteride and protease inhibitors, or combinations thereof.

5. A process according to claim 1, wherein the benefit agent is an anti dandruff agent selected from salicylic acid, sulfur based compositions, keratinization regulators, tar based compositions, steroids, selenium sulfide imidazole antifungal agents, hydroxypyridones and naturopathic agents, or combinations thereof.

6. A process according to claim 1, wherein:
a) the mouth of the first nozzle of the device has an opening of 0,2-3,5 mm²;
b) the mouth of the first nozzle of the device is less than 1 mm away from the wall of the second nozzle.

7. A process according to claim 1, wherein the device further comprises an applicator head.

8. A process according claim 7, wherein the applicator head comprises elements selected from bristles, comb elements, scrub elements, and/or massaging elements.

9. A process according to claim 1, wherein the device is hand-held and connected to a separate unit comprising a compressor and a reservoir for holding a liquid, the compressor being the source of compressed air and the reservoir holding the liquid being the liquid source.

10. A process according to claim 1, wherein the liquid feed source comprises a hair or scalp treatment agent selected from Minoxidil, Finasteride and protease inhibitors, or combinations thereof.

11. A process according to claim 1, wherein the liquid feed source comprises an anti dandruff agent selected from: salicylic acid, sulfur based compositions, keratinization regulators, tar based compositions, steroids, selenium sulfide imidazole antifungal agents, hydroxypyridones and naturopathic agents, or combinations thereof.
